(19) United States
(12) Patent Application Publication
(10) Pub. No.: US 2019/0160286 A1
(43) Pub. Date: May 30, 2019

(54) APPARATUS AND USES THEREOF

(71) Applicant: NATIONAL CHENG KUNG UNIVERSITY, Tainan City (TW)

(72) Inventors: Yen-Kuang YANG, Tainan City (TW); Che-Wei LIN, Tainan City (TW); Shih-Hsien LIN, Tainan City (TW); Kao-Chin CHEN, Tainan City (TW); Ya-Wen CHANG, Tainan City (TW); Ya-Hsin HSIAO, Tainan City (TW)

(73) Assignee: NATIONAL CHENG KUNG UNIVERSITY, Tainan City (TW)

(21) Appl. No.: 16/200,671

(22) Filed: Nov. 27, 2018

Related U.S. Application Data
(60) Provisional application No. 62/590,707, filed on Nov. 27, 2017.

(51) Int. Cl. A61N 1/36; A61B 5/00; G16H 20/40

(52) U.S. Cl. CPC A61N 1/36025; A61B 5/4836; A61B 5/14551; G16H 20/40; A61N 1/36031

(57) ABSTRACT

Disclosed herein is a novel apparatus and the uses thereof in the prophylaxis and/or treatment of neuropsychiatric disorders. The present apparatus comprises a detecting means, a stimulation means, a virtual reality means and a processor. According to some embodiments of the present disclosure, the present apparatus produces an additive or synergistic effect on the treatment of neuropsychiatric disorders.

### CROSS-REFERENCE TO RELATED APPLICATIONS

This application relates to and claims the benefit of U.S. Provisional Application No. 62/590,707, filed Nov. 27, 2017; the content of the application is incorporated herein by reference in its entirety.

### BACKGROUND OF THE INVENTION

### 1. Field of the Invention

The present disclosure in general relates to the field of disease treatment. More particularly, the present disclosure relates to a novel apparatus and the uses thereof in the prophylaxis and/or treatment of neuropsychiatric disorders.

### 2. Description of Related Art

Neuropsychiatric disorders are mental or emotional disorders that arise as a result of underlying diseases or conditions affecting the patient's nervous system. In general, the neuropsychiatric disorders may be classified into four groups: (1) the disorders of thinking and cognition, for example, schizophrenia and delirium; (2) the disorders of mood, such as affective disorders and anxiety; (3) the disorders of social behavior, such as character defects and personality disorders; and (4) the disorders of learning, memory and intelligence, including mental retardation and dementia.

Neuropsychiatric disorders severely compromise the well-being of those affected, with their negative effects on general health and on the ability of children to learn and of adults to work. These disorders have a relatively high prevalence that may have an early onset (for example, autism in childhood and schizophrenia in young adulthood) or a relapsing-remitting course (as in mood and anxiety disorders and compulsive disorder), and often have disabling symptoms. Mental health surveys carried out in the United States suggest that during any 1-year period, approximately 26% of the population will have a mental disorder, and almost 50% of all people will have mental illness sometime during their lifetime. Moreover, it is reported that severe neuropsychiatric conditions have been estimated to occur in 15 to 25% of older adults worldwide.

Neuropsychiatric disorders are complex, heterogeneous conditions resulting from the interaction of factors including genetic, neurobiological, cultural factors and life experiences. Understanding the pathophysiology of neuropsychiatric disorders is challenging due to the inherent complexity of the human brain and the limited types of experimental methodologies that can be applied in human studies. Nowadays, available medications and non-pharmaceutical treatments are merely effective in treating specific symptoms for subsets of affected individuals. However, a significant proportion of individuals with mental disorders do not demonstrate considerable life improvement with available treatments. In addition, serious side effects limit the use of some otherwise effective medications.

In view of the foregoing, there exists in the related art a need for a novel method for preventing and/or treating neuropsychiatric disorders so as to improve the life quality and/or life span of the patients.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to an apparatus for preventing and/or treating a neuropsychiatric disorder in a subject in need thereof. According to embodiments of the present disclosure, the apparatus comprises a detecting means, a stimulation means and a virtual reality (VR) means, in which the detecting means is configured to determine a physiological parameter of the subject, the stimulation means is configured to deliver an electrical pulse to the subject, and the VR means is configured to provide a VR environment to the subject. The processor coupled to the detecting means, the stimulation means and the VR means is configured to alter the electrical pulse and/or the VR environment based on the physiological parameter determined by the detecting means.

According to some embodiments of the present disclosure, the physiological parameter is selected from the group consisting of, heart rate (HR), heart rate variability (HRV), respiratory rate, blood pressure, body temperature, blood oxygen level, electroencephalogram (EEG), electrocorticogram (ECOG), electrocardiogram (ECG) morphology, electrodermal activity (EDA), electromyography (EMG), neuronal activity, and a combination thereof.

In general, the neuronal activity may be determined by evaluating the expression level or concentration of a neurotransmitter selected from the group consisting of, glutamate, γ-Aminobutyric acid (GABA), glutamine, aspartate, serine, glycine, nitric oxide (NO), carbon monoxide (CO), dopamine, norepinephrine, epinephrine, histamine, serotonin, phenethylamine, methylphenethylamine, tyramine, 3-iodothyronamine, octopamine, tryptamine, somatostatin, substance P, opioid peptide, adenosine triphosphate (ATP), adenosine, acetylcholine, and anandamide.

According to certain embodiments, the stimulation means comprises one or more electrodes or coils that are configured to transmit the electrical pulse to the brain of the subject thereby enhancing the neuroplasticity of the subject. In one working example, the stimulation means is a transcranial magnetic stimulation (tMS) device or a transcranial electrical stimulation (tES) device. According to certain embodiments of the present disclosure, the stimulation means is a tES.

In general, the processor is configured to alter the current, voltage, frequency, interpulse interval, position, waveform and/or duration of the electrical pulse delivered by the stimulation means.

Optionally, the VR means may be configured into a headset for providing a visual, auditory and/or olfactory sensations to the subject.

Depending on desired purposes, the process is configured to alter the visual, auditory and/or olfactory sensations provided by the VR.

The second aspect of the present disclosure pertains to a method of preventing and/or treating a neuropsychiatric disorder in a subject in need thereof. The method comprises the steps of,

(a) connecting the present apparatus to the subject;

(b) determining a physiological parameter of the subject; and

(c) based on the physiological parameter determined in the step (b), altering the electrical pulse and the VR environment respectively delivered and provided to the subject via the stimulation means and the VR means.

According to the embodiments of the present disclosure, in the step (a), the stimulation means is connected to the subject so as to deliver electrical pulse to the primary motor cortex, the supplementary motor cortex, the frontal lobe and/or the parietal lobe of the subject.

According to some embodiments, in the step (a), the VR means is configured into a headset, and is worn by the subject, so that a visual, auditory and/or olfactory sensations are provided to the subject.

According to some embodiments, in the step (c), the current, voltage, frequency, interpulse interval, position, waveform and/or duration of the electrical pulse is altered.

The examples of neuropsychiatric disorders treatable with the present apparatus and/or method include, but are not limited to, schizophrenia, delirium, psychotic disorder, dementia, cognitive impairment, benign forgetfulness, closed head injury, autistic spectrum disorder, attention deficit hyperactivity disorder, obsessive compulsive disorder, tic disorder, childhood learning disorder, premenstrual syndrome, depression, bipolar disorder, anxiety disorder, post-traumatic stress disorder, chronic pain, eating disorder, addiction disorder, affective disorder, character defect, personality disorder, Alzheimer's disease, Parkinson's disorder, Huntington's disorder, amyotrophic lateral sclerosis, and a combination thereof.

The subject treatable with the present apparatus and/or method is a mammal; preferably, a human.

Many of the attendant features and advantages of the present disclosure will becomes better understood with reference to the following detailed description considered in connection with the accompanying drawings.

### BRIEF DESCRIPTION OF THE DRAWINGS

The present description will be better understood from the following detailed description read in light of the accompanying drawings, where:

FIG. 1 is a schematic diagram depicting the present apparatus according to one embodiment of the present disclosure.

FIG. 2 is a flowchart depicting the procedure of operating the present apparatus according to one embodiment of the present disclosure.

FIG. 3 is a flowchart depicting the optimizing procedure of the present processor according to one embodiment of the present disclosure.

FIG. 4 is a flow chart depicting the optimizing procedure of the present processor according to another embodiment of the present disclosure.

In accordance with common practice, the various described features/elements are not drawn to scale but instead are drawn to best illustrate specific features/elements relevant to the present invention. Also, like reference numerals and designations in the various drawings are used to indicate like elements/parts.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### I. Definition

For convenience, certain terms employed in the specification, examples and appended claims are collected here. Unless otherwise defined herein, scientific and technical terminologies employed in the present disclosure shall have the meanings that are commonly understood and used by one of ordinary skill in the art. Also, unless otherwise required by context, it will be understood that singular terms shall include plural forms of the same and plural terms shall include the singular. Specifically, as used herein and in the claims, the singular forms “a” and “an” include the plural reference unless the context clearly indicates otherwise. Also, as used herein and in the claims, the terms “at least one” and “one or more” have the same meaning and include one, two, three, or more.

The term “neuropsychiatric disorder” as used herein is intended to refer broadly to any disorder of emotional, personality, and/or mental function that is of neurological origin, psychiatric origin, psychological origin, or mixed origin that negatively impacts the emotional and/or cognitive functioning of a subject. Representative neuropsychiatric disorders include those listed in the Diagnostic and Statistical Manual of Mental Disorders (DSM; including DSM-IV-TR and DSM-5). More particularly, the term “neuropsychiatric disorder” includes, but is not limited to such exemplary conditions as substance use disorders (e.g., use, abuse, and/or dependence on cocaine, opioid, cannabis, amphetamine, alcohol, caffeine, tobacco/nicotine, hallucinogens); anxiety disorders (e.g., post-traumatic stress disorder, obsessive compulsive disorder, panic disorder, agoraphobia, social phobia, acute stress disorder, generalized anxiety disorder, substance-induced anxiety disorder); mood disorders (e.g., both depressive and manic disorders including but not limited to major depressive disorder, major depressive disorder with psychotic features, major depressive disorder with postpartum onset, dysthymic disorder, bipolar I disorder, bipolar II disorder, cyclothymic disorder, substance-induced mood disorder); psychotic disorders (e.g., schizophrenia, schizoaffective disorder, delusional disorder, brief psychotic disorder, shared psychotic disorder, psychotic disorder due to a medical condition, substance-induced psychotic disorder, psychotic disorder not otherwise specified); cognitive disorders (e.g., mild cognitive impairment, Alzheimer's disease, vascular dementia, dementia due to other medical conditions, dementia due to multiple etiologies, substance-induced persisting amnestic disorder, amnestic disorder not otherwise specified, delirium). In some embodiments, the neuropsychiatric disorder is selected from the group consisting of schizophrenia, schizoaffective disorder, Alzheimer's disease, Attention Deficit Disorder/Attention Deficit Hyperactivity Disorder, depression, bipolar disorder, post-traumatic stress disorder (PTSD), a pain disorder, tobacco dependence, alcohol abuse, alcohol dependence, drug dependence, drug abuse, neurodegenerative disorders, sleep disorders, traumatic brain injury and/or concussion, and combinations thereof.

As used herein, the term “determine,” “determining” and “determination” are used interchangeably, and may include measure, calculate, compute, estimate, approximate, generate, and/or otherwise derive, and/or any combination thereof.

As used therein, the term “virtual reality” or “VR” is consistent with its conventional definition, and refers to the computer-simulated environment that can simulate physical presence in places in the real world or imagined worlds. VR could recreate sensory experiences, including virtual taste, sight, smell, sound, touch, and the like.

The term “virtual reality environment” or “VR environment” as used herein should be interpreted broadly to include any real-world or imagined-world environment, in which a subject can feel and/or interact with elements of a three-dimensional (3D) virtual, auditory, tactile, olfactory and/or gustatory displays.

As used herein, the term “treat,” “treating” and “treatment” are interchangeable, and encompasses partially or completely preventing, ameliorating, mitigating and/or managing a symptom, a secondary disorder or a condition associated with neuropsychiatric disorders. The term “treating” as used herein refers to application or administration of the present apparatus to a subject, who has a symptom, a secondary disorder or a condition associated with neuropsychiatric disorders, with the purpose to partially or completely alleviate, ameliorate, relieve, delay onset of, inhibit progression of, reduce severity of, and/or reduce incidence of one or more symptoms, secondary disorders or features associated with neuropsychiatric disorders. Symptoms, secondary disorders, and/or conditions associated with neuropsychiatric disorders include, but are not limited to, anxiety, insomnia, neurotic complaint, apathy, mood disorder, hallucinations, delusions, behavioral and personality changes, delirium, and cognitive impairment (dementia). Treatment may be administered to a subject who exhibits only early signs of such symptoms, disorder, and/or condition for the purpose of decreasing the risk of developing the symptoms, secondary disorders, and/or conditions associated with neuropsychiatric disorders. Treatment is generally “effective” if one or more symptoms or clinical markers are reduced as that term is defined herein. Alternatively, a treatment is “effective” if the progression of a symptom, disorder or condition is reduced or halted.

The term “prevent,” “preventing” and “prophylaxis” as used herein are interchangeable, and refers to the prophylactic treatment of a subject who is at risk of developing a symptom, a secondary disorder or a condition associated with neuropsychiatric disorders, so as to decrease the probability that the subject will develop the symptom, secondary disorder or condition. Specifically, the term “prevent,” “preventing” or “prophylaxis” refers to inhibit the occurrence of a symptom, a secondary disorder or a condition associated with neuropsychiatric disorder, that is to reduce the incidence or the frequency of occurrence of the symptom, secondary disorder or condition. The term “prevent,” “preventing” or “prophylaxis” as used herein referring to the present apparatus and/or method, does not mean or imply that use of the present apparatus and/or method will provide a guarantee that the symptom, secondary disorder or condition will never occur, but rather that the present apparatus and/or method will inhibit the occurrence of the symptom, secondary disorder or condition, and that the incidence and/or frequency of the symptom, secondary disorder or condition will be reduced.

The term “additive effect” as used herein refers to the combined effect of two or more treatments (e.g., the electrical pulse delivered by the present stimulation means, and the VR environment provided by the present VR means) that is approximately equal to the sum of the effect of each treatment given alone.

The term “synergistic effect” as used herein refers to action of two or more treatments (e.g., the electrical pulse delivered by the present stimulation means, and the VR environment provided by the present VR means) producing an effect, for example, preventing, slowing and/or treating the development and/or progress of a neuropsychiatric disorder or symptoms thereof, which is greater than the simple addition of the effects of each treatment administered by themselves. A synergistic effect can be calculated, for example, using suitable methods such as the Sigmoid-Emax equation (Holford, N. H. G. and Scheiner, L. B., Clin. Pharmacokinet 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the treatment combination. The corresponding graphs associated with the equations referred to above are the concentration-effect curve, isobologram curve and combination index curve, respectively.

The term “subject” or “patient” refers to an animal including the human species that is treatable with the apparatus and/or methods of the present disclosure. The term “subject” or “patient” intended to refer to both the male and female gender unless one gender is specifically indicated. Accordingly, the term “subject” or “patient” comprises any mammal which may benefit from treatment of neuropsychiatric disorders. Examples of a “subject” or “patient” include, but are not limited to, a human, rat, mouse, guinea pig, monkey, pig, goat, cow, horse, dog, cat, bird and fowl. In an exemplary embodiment, the patient is a human.

### II. Description of the Invention

The present disclosure is directed to an apparatus for preventing and/or treating a neuropsychiatric disorder in a subject in need thereof for example, the subject having a risk of developing a neuropsychiatric disorder, or the subject having or suspected of having a neuropsychiatric disorder.

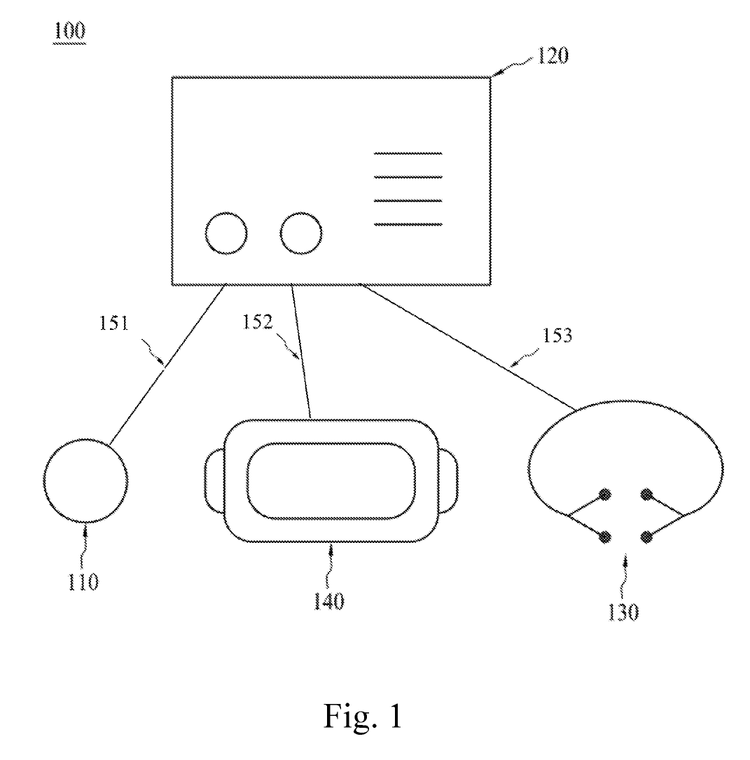

Reference is now made to FIG. 1, which is a schematic diagram depicting the present apparatus 100 in accordance with embodiments of the present disclosure. In structure, the apparatus 100 comprises a detecting means 110, a processor 120, a stimulation means 130, and a VR means 140, in which the detecting means 110, the stimulation means 130, and the VR means 140 are respectively coupled to the processor 120.

As exemplified in FIG. 1, the detecting means 110, the stimulation means 130 and the VR means 140 may be respectively coupled to the processor 120 by light guides (151, 152, 153). Optionally, each of the light guides (151, 152, 153) is sheathed in a material that is any of plastic, resin, glass, ceramic, or a combination thereof (e.g., forming a plastic-, resin-, glass-, ceramic- or plastic glass-covered coil). Examples of the plastic suitable for sheathing the light guides (151, 152, 153) include, but are not limited to, polyvinyl chloride (PVC), polyethylene (PE), polypropylene (PP), polystyrene (PS), polyethylene terephthalate (PET), polyvinyl acetate (PVAc), vinyl acetate (VA), and a combination thereof. The resin may be a plant resin (i.e., natural resin; such as amber, guaiac resin, copal, kauri gum, dammar, mastic, sandarac, and etc.), or a synthetic resin (e.g., epoxy resin, polyester resin and acetal resin). The glass may be made of silica, boron, phosphate, aluminum, chalcogen element, fluoride, or a combination thereof. Exemplary material for producing the ceramic include, aluminum oxide, zirconium oxide, silicon carbide, silicon nitride, and a combination thereof.

Alternatively, the detecting means 110, the stimulation means 130 and the VR means 140 may be respectively coupled to the processor 120 via a wireless connection, such as, bluetooth, wireless fidelity (WiFi), infrared, ultra-wideband connection, and the like. The wireless coupling is also within the scope of the present disclosure.

The detecting means 110 is configured to determine one or more (e.g., two, three, four, five or more) physiological parameters of the subject; for example, HR, HRV, respiratory rate, blood pressure, body temperature, blood oxygen level, EEG, ECOG, ECG morphology, EDA, EMG, neuronal activity, and/or a combination thereof. The physiological parameter(s) may be determined by conventional technique; for example, the neuronal activity may be determined by EEG and/or evaluating the expression level/concentration of a neurotransmitter. The methods for evaluating/determining the expression level of a neurotransmitter include, but are not limited to, magnetic resonance spectroscopy (MRS), position emission tomography (PET), single photon imaging computed tomography (SPECT), magnetic resonance imaging (MRI), computed axial X-ray tomography (CAT), and a combination thereof.

As would be appreciated, the neurotransmitter may be any endogenous molecule that transmits signals from one neuron to another neuron. The neurotransmitter may be excitatory or inhibitory molecule. Depending on intended purposes, the neurotransmitter may be glutamate, GABA, glutamine, aspartate, serine, glycine, NO, CO, dopamine, norepinephrine, epinephrine, histamine, serotonin, phenethylamine, methylphenethylamine, tyramine, 3-iodothyronamine, octopamine, tryptamine, somatostatin, substance P, opioid peptide, ATP, adenosine, acetylcholine. or anandamide. In some working examples of the present disclosure, the neurotransmitter is glutamate or GABA.

According to certain embodiments of the present disclosure, the physiological parameter is blood pressure (i.e., systolic blood pressure and/or diastolic blood pressure). According to alternative embodiments, the physiological parameter is HR. According to some embodiments, the physiological parameter is HRV. In alternative examples, the physiological parameter is the expression level/concentration of a neurotransmitter (for example, glutamate or GABA).

The stimulation means 130 is configured to deliver an electrical pulse to the subject. Depending on desired purposes, the stimulation means 130 may comprise one or more (e.g., two, three, four, five or more) electrodes or coils, which are configured to transmit the electrical pulse to the brain of the subject. According to some embodiments of the present disclosure, the electrical pulse is useful in enhancing the neuroplasticity of the subject. Preferably, the stimulation means 130 is a transcranial magnetic stimulation (tMS) device or a transcranial electrical stimulation (tES) device. According to some working examples, the stimulation means 130 is a tES device, in which non-invasive technique is employed to target brain regions using arrays of electrodes on the scalp.

Preferably, the detecting means 110 and the electrode of the stimulation means 130 are respectively configured in the form of pads. Each pad is composed of, from top to bottom, a releasing film, an adhesive layer, and a supporting substrate, in which the detecting means 110 or the electrode of the stimulation means 130 is fixed on the supporting substrate of the pad by the adhesive layer. During operation, a user tears away the releasing film to expose the detecting means 110 or the electrode of the stimulation means 130 fixed on the supporting substrate by the adhesive layer, then the pad is secured to the intended site (e.g., the head, neck, chest, limb and abdomen etc.) by pressing the side of the adhesive layer against the target site. According to the embodiments of the present disclosure, the pad may be made of any conventional material, preferably, made of resilient polyurethane, natural or synthetic rubber, or fabric.

Alternatively, each of the detecting means 110 and stimulation means 130 may be configured into a wearable device to be worn by the subject, for example, each of the detecting means 110 and the stimulation means 130 may be configured into a headset, a bracelet, a necklace, a ring, a belt, a band, a garment, or shoes.

The VR means 140 is configured to provide a VR environment to the subject. According to the preferred embodiment, the VR means 140 is configured into a headset, which provides a visual, auditory and/or olfactory sensations to the subject that is different from his/her actual physical environment. Specifically, the headset may comprise an ocular mask and one or more conduits so as to provide the subject with a VR experience to visualize, hear and/or smell a three-dimensional (3D) design.

According to alternative embodiments, the VR means 140 comprises a headpiece having at least one 3D VR electronic display, which can be used in the head section; at least one sound generator, which provides 3D virtual sound coordinated with the 3D VR electronic display; and optionally, at least one olfactory providing mechanism, which delivers olfactory chemicals to stimulate the olfactory sense of the subject.

The processor 120 is configured to alter/modify the electrical pulse delivered by the stimulation means 130, as well as the VR environment provided by the VR means 140. According to the embodiments of the present disclosure, the alteration/modification is performed based on the physiological parameter determined by the detecting means 110.

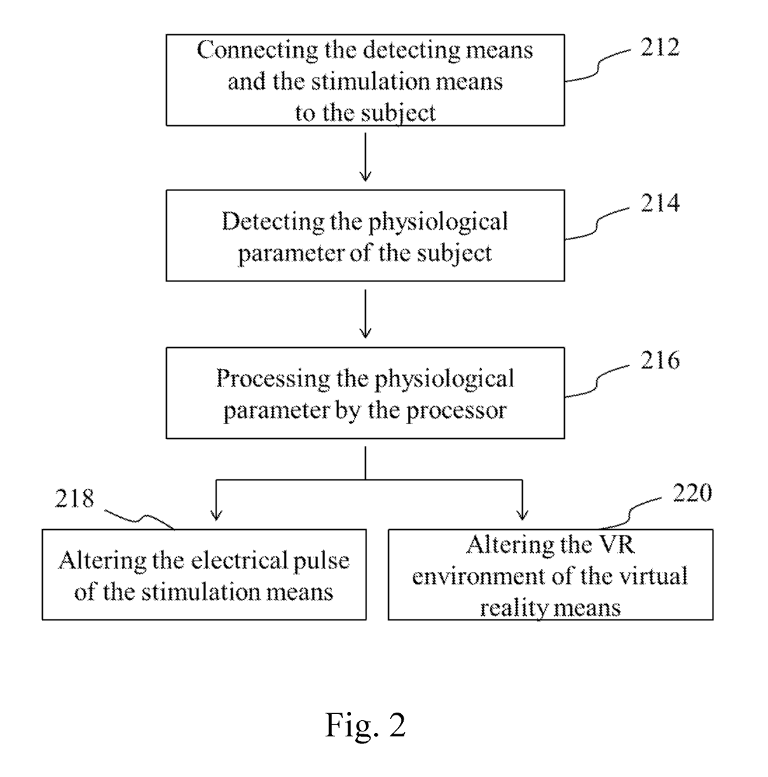

Reference is now made to FIG. 2, which depicts the procedure for the treatment of neuropsychiatric disorder by use of the present apparatus 100. In step 212, the detecting means 110 and the electrodes or coils of the stimulation means 130 are respectively placed at suitable sites in accordance with the desired therapeutic effect. For example, the detecting means 110 may be disposed on the head, neck, chest, limb and abdomen of the subject so as to detect the physiological parameter. The electrodes or coils of the stimulation means 130 may be disposed on the forehead, the frontal part and/or the anterior part of the head of the subject thereby delivering the electrical pulse to the primary motor cortex, the supplementary motor cortex, the frontal lobe and/or the parietal lobe of the subject.

The present apparatus is characterized in that the electrical pulse and the VR environment delivered/provided to the subject are altered/modified in accordance with his/her physical condition. Specifically, as depicted in steps 214 and 216 of FIG. 2, one or more physiological parameter(s) of the subject is/are detected by the detecting means 110 followed by being transmitted to and processed by the processor 120. Then, the processor 120 in turn transmits a first signal to the stimulation means 130 and/or a second signal to the VR means 140 thereby altering/modifying the electrical pulse (e.g., the current, voltage, frequency, interpulse interval, position, waveform and/or duration of the electrical pulse) and/or the VR environment (e.g., the visual, auditory and/or olfactory environment) based on the subject's physical attributes (steps 218 and 220 of FIG. 2).

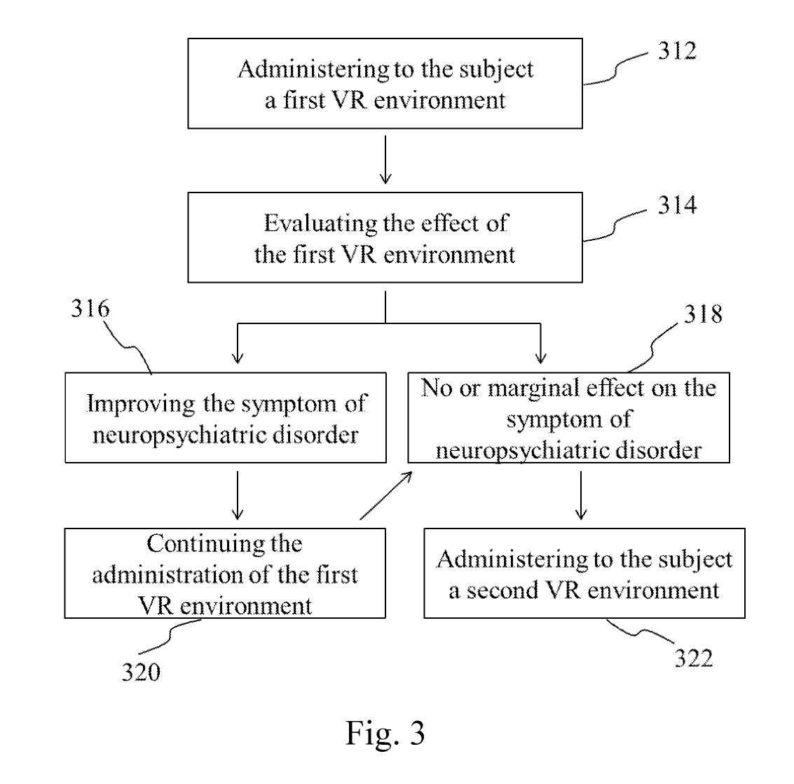

The processor 120 is useful in optimizing the parameter/condition of the electrical pulse and/or the VR environment in accordance with the physical condition detected. FIG. 3 provides an exemplary optimizing procedure, in which a first VR environment (e.g., one delightful environment) is administered to the subject having a neuropsychiatric disorder (step 312), and the effect of the first VR environment on the subject is evaluated by the detecting means 110 (step 314). In the case when the first VR environment improves the symptoms of the neuropsychiatric disorder (step 316; e.g., increasing HRV, increasing the expression level/concentration of GABA, and/or reducing the expression level/concentration of glutamate), then the administration of the first VR environment continues (step 320). Alternatively, when the first VR environment exhibits no or marginal effect on the symptoms of the neuropsychiatric disorder (step 318), then the processor alters the treatment by replacing the first VR environment with a second VR environment (e.g., another delightful environment or a meditative environment) (step 322). Optionally, the image, sound and/or olfactory chemicals of the second VR environment is adjusted by the processor in accordance with the physical parameters (e.g., HRV, and/or the expression level/concentration of GABA or glutamate) of the subject.

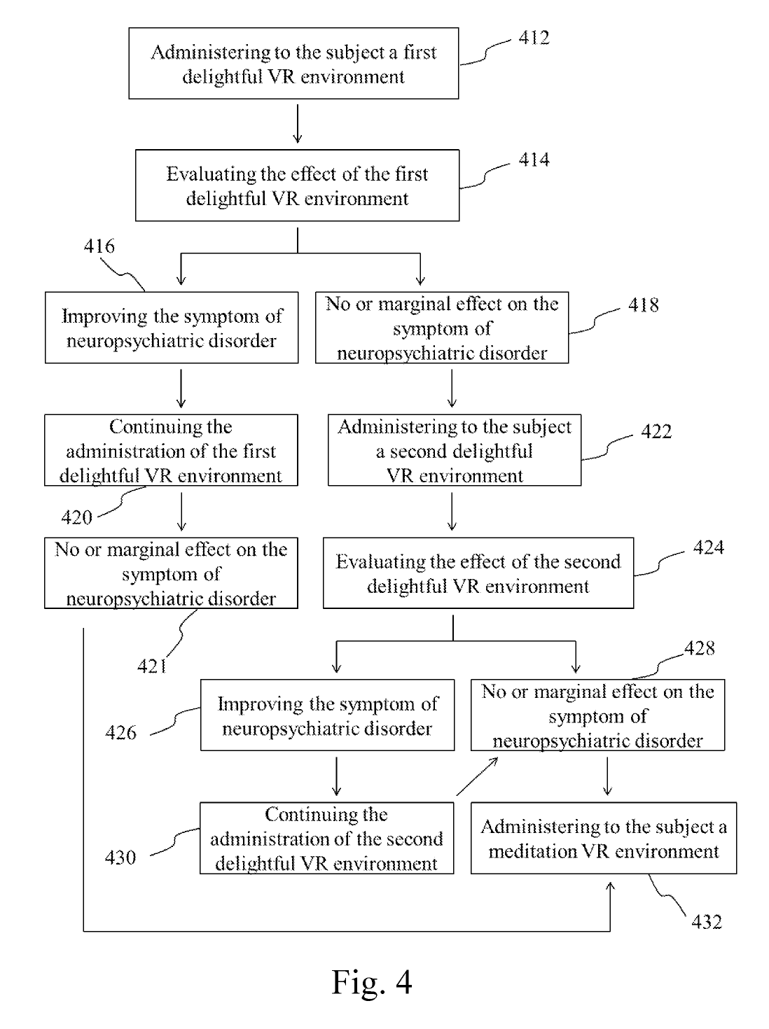

FIG. 4 provides a working example of the optimizing procedure, in which three VR environments are prepared for the treatment of the subject having a neuropsychiatric disorder. Steps 412 to 422 are similar with steps 312 to 322 of FIG. 3, and hence, detailed description thereof is omitted herein for the sake of brevity. According to FIG. 4, both the first and second VR environments as illustrated in steps 412 and 422 are delightful VR environments (i.e., the first and second delightful VR environments). In the case when the administration of the first or second delightful VR environment exhibits therapeutic effect on neuropsychiatric disorder (steps 416 and 426), then it continues (steps 420 and 430). When the subject produces a tolerance response toward the treatment (i.e., being not responsive to the first or second delightful VR environment) (steps 421 and 428), then the delightful VR environment is replaced by a meditation VR environment (step 432). As mentioned above, the image, sound and/or olfactory chemicals of the meditation VR environment may be adjusted by the processor in accordance with the physical parameters (e.g., HRV, and/or the expression level/concentration of GABA or glutamate) of the subject.

As would be appreciated, the VR environment may vary with the conditions of the subject. In addition to the delightful and meditative environments as exemplified in FIGS. 3 and 4, the clinical practitioner or the skilled artisan may choose any VR environment as long as it improves the emotional state (e.g., the perception of relaxation, calmness, ease, happiness, energy and other positive emotions) of the subject, for example, an ethereal, dreamlike, safe, relaxing, and cheerful VR environments. Alternatively, the skilled artisan may design a customized VR environment based on the desired purposes.

In general, the parameter(s) (e.g., the current, voltage, frequency, interpulse interval, position, waveform and/or duration) of the electrical pulse delivered by the stimulation means 130 is optimized by the processor 120 in a similar manner, in which a starting electrical pulse is first administered to the subject having a neuropsychiatric disorder, and the physical parameter(s) thereof is monitored by the detecting means 110. The starting electrical pulse is maintained until the symptom of the neuropsychiatric disorder is no longer improved, and then, a second electrical pulse is provided to the subject. The starting electrical pulse and the second electrical pulse may vary with the condition of the patients. Preferably, 5-50 mA (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mA) of the starting electrical pulse and/or the second electrical pulse is administered to the subject for 5-50 minutes (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 minutes). More preferably, 10-30 mA of the starting electrical pulse and/or the second electrical pulse is administered to the subject for 10-30 minutes. In one working example of the present disclosure, 20 mA of the starting electrical pulse and/or the second electrical pulse is administered to the subject for 20 minutes. The stimulation of the electrical pulse may be am anodal stimulation or a cathodal stimulation.

The processor 120 is configured to optimize the VR environment and the electrical pulse thereby providing a synergistically or additively therapeutic effect on neuropsychiatric disorders, for example, synergistically or additively alleviating or ameliorating one or more symptoms associated with the neuropsychiatric disorder.

According to the embodiments of the present disclosure, the electrical pulse is useful in preventing and/or treating a neuropsychiatric disorder in a subject in need thereof (e.g., the subject having a risk of developing a neuropsychiatric disorder, or the subject having or suspected of having a neuropsychiatric disorder); and the VR environment improves the emotional state of the subject that in turn enhances the therapeutic effect of the electrical pulse. In these embodiments, the stimulation means 130 and the VR means 140 produce an additive/synergistic effect on the treatment of neuropsychiatric disorders.

Non-limiting examples of the neuropsychiatric disorders treatable with the present apparatus and/or method include, schizophrenia, delirium, psychotic disorder, dementia, cognitive impairment, benign forgetfulness, closed head injury, autistic spectrum disorder, attention deficit hyperactivity disorder, obsessive compulsive disorder, tic disorder, childhood learning disorder, premenstrual syndrome, depression, bipolar disorder, anxiety disorder, post-traumatic stress disorder, chronic pain, eating disorder, addiction disorder, affective disorder, character defect, personality disorder, Alzheimer's disease, Parkinson's disorder, Huntington's disorder, amyotrophic lateral sclerosis, and a combination thereof.

The subject treatable with the present apparatus and/or method is a mammal, for example, a human, a mouse, a rat, a hamster, a guinea pig, a rabbit, a dog, a cat, a cow, a goat, a sheep, a monkey, and a horse. Preferably, the subject is a human.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### Example

Patients

The patients suffered from anxiety and poor sleep were enrolled in the present investigation. This investigation was approved by the Institutional Review Board. Written informed consent was obtained from each patients.

To evaluate the therapeutic effect of the present apparatus on anxiety, the HRV (it is known that reduced HRV is associated with anxiety) and the sleeping quality of the patients were evaluated before and after the treatment. In the beginning, a transcranial direct current stimulation (tDCS; 20 mA for 20 minutes) was administered to the patient, in which one electrode of the tDCS was disposed on the left-hand side of the frontal part of the head of the patient, while the other electrode of the tDCS was disposed on the right-hand side of the frontal part of the head of the patient. The detecting means was placed on wrist of the patient so as to detect the HRV thereof. The HRV signals were transmitted to the processor, which then adjusted the treatment to be administered based on the received HRV signals. In the case when the tDCS did not increase the HRV of the patient, an interactive VR was co-administered with the tDCS to the patient. The VR was provided by a headset, which delivered a delightful visual and auditory sensation to the patient. When the co-administration of tDCs and a first selected VR increased the HRV of the patient, then continued playing the first selected VR; by contrast, in the case when such a co-administration did not obviously change the HRV of the patient, then the first selected VR was replaced by a second selected VR, which delivered another delightful visual and auditory sensation to the patient. The selected VR and tDCS treatments were co-administered to the patients once per day until no increase in HRV was observed.

Then, in the second round of treatment, a tDCS (20 mA for 20 minutes) was administered to the patient once, followed by co-administration with an interactive VR. The treatment procedure of the second round was similar to that of the first round of treatment, except for the interactive VR providing a meditative condition (i.e., a peaceful meditative environment), instead of the delightful condition. The results of one representative patient were summarized in Tables 1-3.

### Example 1 the Effect of the Present Apparatus on Anxiety

The systolic blood pressure (SYS), diastolic blood pressure (DIA), HR and HRV of the patient before and after the treatment were summarized in Table 1. The data indicated that the co-administration of the iDCS and delightful VR increased the HRV of the patient; however, the patient produced a tolerance response toward the treatment after being subjected to the same treatment for three times. Therefore, the delightful VR was replaced by a meditation VR. After two rounds of treatment, the HRV was obviously elevated in the patient.

TABLE 1
Treatment procedure and physiological parameter of the representative
patient

| | | | HR | | |
|---|---|---|---|---|---|
| | SYS | DIA | (beats/ | | |
| Day | (mmhg) | (mmhg) | minute) | HRV | Event |
| 0 | 119 | 88 | 65 | 44 | Baseline treatment |
| 1 | 121 | 92 | 75 | 59 | before tdcs |
| 1 | 115 | 88 | 67 | 48 | after tdcs |
| 2 | 123 | 92 | 60 | 63 | before tdcs and delightful VR |
| 2 | 133 | 96 | 60 | 51 | after tdcs and delightful VR |
| 3 | 128 | 98 | 61 | 42 | before tdcs and delightful VR |
| 3 | 124 | 90 | 70 | 56 | after tdcs and delightful VR |
| 7 | 127 | 92 | 65 | 51 | before tdcs, VR stopped |
| 7 | 127 | 94 | 63 | 62 | after tdcs, VR stopped |
| 8 | 131 | 96 | 70 | 52 | before TDCS and meditation |
| | | | | | VR |
| 8 | 128 | 96 | 71 | 50 | after TDCS and meditation |
| | | | | | VR |
| 9 | 120 | 90 | 65 | 64 | before TDCS and meditation |
| | | | | | VR |
| 9 | 127 | 90 | 67 | 62 | after TDCS and meditation |
| | | | | | VR |

It has been reported that the expression level of glutamate, a stimulating/activating neurotransmitter, is positively correlated with the level of anxiety; while GABA, an inhibitory neurotransmitter, is useful in reducing anxiety. Thus, in addition to the parameters listed in Table 1, the expression level of GABA and glutamate in the brain of the patient were also examined by MRS. As summarized in Table 2, the data indicated that the present treatment remarkably increased the expression level of GABA.

TABLE 2
The expression level of glutamate and GABA before and after treatment

| | | Before treatment | After treatment |
|---|---|---|---|
| Brain | | (relative expression | (relative expression |
| region | Neurotransmitter | level to creatine)* | level to creatine)* |
| ACC | Glutamate | 15.23 | 21.26 |
| | GABA | 0.495 | 1.54 |
| MPFC | Glutamate | 9.94 | 11.02 |
| | GABA | 0.319 | 1.892 |

ACC: Anterior cingulate cortex.
MPFC: Medial prefrontal cortex.
*the value of glutamate/creatine, or GABA/creatine.

The clinical assessment (with standard instrument for clinical purpose, Hamilton's rating scale for depression, and Hamilton's rating scale for anxiety, performed by senior psychiatrist, who is blind to the purpose of this experiment) further confirmed the effect of the present apparatus on improving the symptoms of anxiety (Table 3).

TABLE 3
Clinical assessment before and after treatment

| | Before | After | |
|---|---|---|---|
| | treatment | treatment | Improvement |
| Depression | | | |
| Depressed mood | 0 | 0 | |
| Feelings of guilt | 0 | 0 | |
| Suicide | 0 | 0 | |
| Insomnia-intial | 0 | 0 | |
| Insomnia-middle | 0 | 0 | |
| Insomnia-delayed | 1 | 0 | V |
| Work and intersts | 0 | 0 | |
| Retardation | 0 | 0 | |
| Agitation | 0 | 0 | |
| Anxiety-pschic | 2 | 1 | V |
| Axiety-somatic | 0 | 1 | X |
| Somatic symptoms-gastrointestinal | 0 | 0 | |
| Somatic symptoms-general | 0 | 0 | |
| Genital Symptoms | 0 | 0 | |
| Hypochondriasis | 0 | 0 | |
| Weight Loss | 0 | 0 | |
| Insight | 0 | 0 | |
| Diurnal variation | 1 | 0 | V |
| Depersonalization | 0 | 0 | |
| Paranoid symptoms | 0 | 0 | |
| Obsessional Symptoms | 0 | 0 | |
| Anxietry | | | |
| Anxious mood | 1 | 1 | |
| Tension | 2 | 1 | V |
| Fears | 0 | 0 | |
| Insomnia | 2 | 0 | V |
| Intellectual | 0 | 0 | |
| Depressed mood | 0 | 0 | |
| Somatic (muscular) | 1 | 0 | V |
| Somatic (sensory) | 0 | 0 | |
| Cardiovascular symptoms | 0 | 0 | |
| Respitatory symptoms | 0 | 0 | |
| Gastrointestinal symptoms | 0 | 0 | |
| Genitourinaty symptoms | 0 | 0 | |
| Autonomic symptoms | 1 | 0 | V |
| Behavior at interview | 1 | 0 | V |

In conclusion, the present disclosure provides an apparatus for preventing and/or treating a neuropsychiatric disorder (e.g., anxiety) in a subject. According to the example of the present disclosure, the present apparatus is useful in alleviating the symptoms associated with anxiety, including improving insomnia, diurnal variation, tension and etc.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. An apparatus for preventing and/or treating a neuropsychiatric disorder in a subject in need thereof, comprising,
a detecting means configured to determine a physiological parameter of the subject;
a stimulation means configured to deliver an electrical pulse to the subject;
a virtual reality means configured to provide a virtual reality environment to the subject; and
a processor coupled to the detecting means, the stimulation means and the virtual reality means, wherein the processor is configured to alter the electrical pulse and/or the virtual reality environment based on the physiological parameter determined by the detecting means.

2. The apparatus of claim 1, wherein the physiological parameter is selected from the group consisting of, heart rate (HR), heart rate variability (HRV), respiratory rate, blood pressure, body temperature, blood oxygen level, electroencephalogram (EEG), electrocorticogram (ECOG), electrocardiogram (ECG) morphology, electrodermal activity (EDA), electromyography (EMG), neuronal activity, and a combination thereof.

3. The apparatus of claim 2, wherein the neuronal activity is determined by evaluating the expression level of a neurotransmitter selected from the group consisting of, glutamate, γ-Aminobutyric acid (GABA), glutamine, aspartate, serine, glycine, nitric oxide (NO), carbon monoxide (CO), dopamine, norepinephrine, epinephrine, histamine, serotonin, phenethylamine, methylphenethylamine, tyramine, 3-iodothyronamine, octopamine, tryptamine, somatostatin, substance P, opioid peptide, adenosine triphosphate (ATP), adenosine, acetylcholine, and anandamide.

4. The apparatus of claim 1, wherein the stimulation means comprises one or more electrodes or coils configured to transmit the electrical pulse to the brain of the subject thereby enhancing the neuroplasticity of the subject.

5. The apparatus of claim 1, wherein the stimulation means is a transcranial magnetic stimulation (tMS) device or a transcranial electrical stimulation (tES) device.

6. The apparatus of claim 5, wherein the processor is configured to alter the current, voltage, frequency, interpulse interval, position, waveform and/or duration of the electrical pulse delivered by the stimulation means.

7. The apparatus of claim 1, wherein the virtual reality means is configured into a headset for providing a visual, auditory and/or olfactory sensations to the subject.

8. The apparatus of claim 7, wherein the processor is configured to alter the visual and/or auditory sensations of the virtual reality means.

9. The apparatus of claim 1, wherein the neuropsychiatric disorder is selected from the group consisting of, schizophrenia, delirium, psychotic disorder, dementia, cognitive impairment, benign forgetfulness, closed head injury, autistic spectrum disorder, attention deficit hyperactivity disorder, obsessive compulsive disorder, tic disorder, childhood learning disorder, premenstrual syndrome, depression, bipolar disorder, anxiety disorder, post-traumatic stress disorder, chronic pain, eating disorder, addiction disorder, affective disorder, character defect, personality disorder, Alzheimer's disease, Parkinson's disorder, Huntington's disorder, amyotrophic lateral sclerosis, and a combination thereof.

10. The apparatus of claim 1, wherein the subject is a human.

11. A method of preventing and/or treating a neuropsychiatric disorder in a subject in need thereof, comprising,
(a) connecting the apparatus of claim 1 to the subject;
(b) determining a physiological parameter of the subject; and
(c) based on the physiological parameter determined in the step (b), altering the electrical pulse and the virtual reality environment respectively delivered and provided to the subject via the stimulation means and the virtual reality means.

12. The method of claim 11, wherein in the step (a), the stimulation means is connected to the subject to deliver the electrical pulse to the primary motor cortex, the supplementary motor cortex, the frontal lobe and/or the parietal lobe of the subject.

13. The method of claim 11, wherein in the step (a), the virtual reality means is configured into a headset, and is worn by the subject, so that a visual, auditory and/or olfactory sensations are provided to the subject.

14. The method of claim 11, wherein the physiological parameter of the step (b) is selected from the group consisting of, heart rate (HR), heart rate variability (HRV), respiratory rate, blood pressure, body temperature, blood oxygen level, electroencephalogram (EEG), electrocorticogram (ECOG), electrocardiogram (ECG) morphology, electrodermal activity (EDA), electromyography (EMG), neuronal activity, and a combination thereof.

15. The method of claim 14, wherein the neuronal activity is determined by the expression of a neurotransmitter selected from the group consisting of, glutamate, γ-Aminobutyric acid (GABA), glutamine, aspartate, serine, glycine, nitric oxide (NO), carbon monoxide (CO), dopamine, norepinephrine, epinephrine, histamine, serotonin, phenethylamine, methylphenethylamine, tyramine, 3-iodothyronamine, octopamine, tryptamine, somatostatin, substance P, opioid peptide, adenosine triphosphate (ATP), adenosine, acetylcholine, and anandamide.

16. The method of claim 11, wherein in the step (c), the current, voltage, frequency, interpulse interval, position, waveform and/or duration of the electrical pulse is altered.

17. The method of claim 11, wherein the neuropsychiatric disorder is selected from the group consisting of, schizophrenia, delirium, psychotic disorder, dementia, cognitive impairment, benign forgetfulness, closed head injury, autistic spectrum disorder, attention deficit hyperactivity disorder, obsessive compulsive disorder, tic disorder, childhood learning disorder, premenstrual syndrome, depression, bipolar disorder, anxiety disorder, post-traumatic stress disorder, chronic pain, eating disorder, addiction disorder, affective disorder, character defect, personality disorder, Alzheimer's disease, Parkinson's disorder, Huntington's disorder, amyotrophic lateral sclerosis, and a combination thereof.

18. The method of claim 11, wherein the subject is a human.
